(19) United States
(12) Patent Application Publication
(10) Pub. No.: US 2008/0305193 A1
(43) Pub. Date: Dec. 11, 2008

(54) Cosmetic compositions

(76) Inventor: James R. Duprey, JR., New York, NY (US)

Correspondence Address: S. Michael Bender, P.O. Box 530-399, St. Petersburg, FL, 33747, US

(21) Appl. No.: 11/809,947

(22) Filed: Jun. 5, 2007

(51) Int. Cl. A61K 36/00; A61K 31/07; A61K 31/195; A61K 31/335; A61K 31/34; A61P 43/00; A61K 31/36; A61K 31/59; A61K 31/70

(52) U.S. Cl. 424/776; 514/167; 514/23; 514/458; 514/474; 514/52; 514/561; 514/725

(57) ABSTRACT

A cosmetic composition suitable for topical application to human or animal skin comprising an effective amount of active ingredient dispersed in an acceptable cosmetic base or carrier, wherein the active ingredient comprises a mixture of an amino acid, a low glycemic glycoside and a Vitamin, wherein the amino acid is L-arginine, and wherein the low glycemic glycoside is derived from kiwi fruit.

### BACKGROUND OF THE INVENTION

1. Field of the Invention

The present invention relates generally to improved cosmetic compositions and more particularly, to cosmetic compositions containing an active ingredient or agent capable of imparting beneficial effects to human or animal skin.

2. Description of the Prior Art

It is well known in the art of formulating cosmetic compositions or preparations to include ingredients that provide enhanced benefits to the skin. For example, in U.S. Pat. No. 4,826,828 (Wilmott et al.), there are disclosed cosmetic compositions including retinol dispersed in the cosmetic base for reducing fine lines and wrinkles. Similarly, in U.S. Pat. No. 6,299,889 (Cowton et al) and U.S. Pat. No. 4,983,382 (Wilmot et al.), there are disclosed stable cosmetic compositions containing ascorbic acid (Vitamin C) for imparting beneficial appearance effects to the skin including improved tone and luster, a decrease in fine lines and wrinkles, and improved elasticity, among other benefits. The contents of the aforementioned U.S. Pat. Nos. 4,826,828; 6,299,889; and 4,983,382 hereby are incorporated herein by this reference.

Amino acids are another class of compound that are capable of imparting benefits to human or animal skin when topically applied in a suitable base or carrier. U.S. Pat. No. 5,204,099 teaches that amino acids selected from the group consisting of arginine, serine, proline, histidine, alanine, lysines, ornithine, citrulline, urocanic acid, aspargain or tyrosine when copolymerized with glutamic acid or aspartic acid produces a molecule having durable moisturizing properties when applied to human skin. U.S. Pat. No. 6,207,713 is yet another example which reports that the amino L-arginine orally or topically applied to humans can produce beneficial effects such as warming, increased blood flow, healing and so on. The contents of the aforementioned U.S. Pat. Nos. 5,204,099 and 6,207,713 hereby also are incorporated herein by this reference.

Unfortunately, not all amino acids, including specifically, arginine, or L-arginine, are suitable for human consumption. In a clinical study published in the Journal of the American Medical Association (JAMA), Vol. 295 No. 1, Jan. 4, 2006, entitled L-Arginine Therapy in Acute Myocardial Infarction, it has been reported that L-arginine consumed by humans over a wide dosage range had adverse effects. Because of the many beneficial effects arginine has on human skin resulting in less drying, anti-aging and collagen support, there is a need to provide cosmetic compositions that include amino acids such as arginine or L-arginine for their beneficial effects, but which are also safe for human or animal use and consumption. This need is met by the present invention which has as its primary object the provision of improved cosmetic compositions having an amino acid ingredient in combination with another ingredient or ingredients which have the unexpected advantageous effect of rendering the amino acid innocuous when applied to human or animal skin.

### SUMMARY OF THE INVENTION

To meet the foregoing need and achieve other objects and advantages, the present invention, briefly described, relates to cosmetic compositions that include an amino acid active ingredient that is rendered safe as well as effective. It has been discovered that when an amino acid component in the form of L-arginine is combined with a blind amino acid rider (BARR) and the combined ingredient added to a suitable cosmetic carrier or base which may be topically applied to human or animal skin, the amino acid is readily absorbed by the skin in a perfectly safe manner with no ill side effects. In accordance with the invention, the mostly preferred blind amino acid rider is a low glycemic naturally occurring glycoside derived from the kiwi fruit and made commercially available under the designation TRUTINA DULCEM by Nutrilab Corporation, St. Petersburg, Fla. The suitable cosmetic base or carrier in which the active ingredient according to the present invention is contained may be any well known form such as for example, skin lotion, makeup, lipstick or lip color and so on. Thus, in its broadest aspect It has been found in accordance with invention that a glycosidic agent having a low glycemic index may be combined with an amino acid and other optionally desirable cosmetically acceptable additives to produce stable cosmetic compositions that are safe and effective.

Although the foregoing brief description sets forth rather broadly the more important features of the present invention in order that the detailed description thereof that follows may be better understood, and in order that the present contributions to the art may be better appreciated, there are, of course, additional features, objects and advantages of the invention that will be described hereinafter and which will be for the subject matter of the claims appended hereto.

### DETAILED DESCRIPTION OF THE PREFERRED EMBODIMENT

In accordance with the invention, an active ingredient comprising at least three components, namely an amino acid, a low glycemic glycoside and Vitamin C, are added to a cosmetically acceptable base or carrier to yield an improved stable cosmetic composition adapted to be topically applied to human or animal skin whereby the active ingredient imparts beneficial effects to the skin surface including resistance to aging, improved tone and luster, moisture retention, collagen nourishment and support, and wrinkle effacement, among others.

The mostly preferred amino acid suitable for the cosmetic compositions of the present invention is L-arginine which is more fully described in U.S. Pat. No. 6,608,109 entitled Composition Comprising L-Arginine As A Muscle Growth Stimulant And Use thereof, the contents of which patent are incorporated herein by this reference. Arginine is the amino acid always found in protein food substances and always in combination with its sister amino acids. L-arginine is a free form amino acid separated from other amino acids by fermentation as is well known in the art. The L-arginine used in the present invention is the base form described in column 3, lines 31 et seq. of the aforementioned '109 patent (as opposed to the acid salt HCL). The amount of L-arginine used in the cosmetic preparations of the present invention will vary according to the effect desired. Generally, L-arginine 20 is used in an amount ranging between about 0.05% to about 3.0% of the total weight of the composition with an amount of about 0.75% being especially preferred.

In accordance with the invention, and in order to render the L-arginine safe as well as effective in the cosmetic compositions of the invention, each L-arginine molecule is co-bonded to a chemical rider in the form of a low glycemic glycoside derived from the kiwi fruit and in this regard, the L-arginine and the glycoside preferably mixed together in a 1:1 ratio by weight. The low glycemic glycoside derived from kiwi fruit as contemplated by the present invention is said to function as a Blind Amino Acid™ rider (BARR) rendering the L-arginine non-toxic when absorbed percutaneously by the skin. In accordance with the invention, the glycoside particles are “nano-sized” as this facilitates binding with the L-arginine molecule. By “nano-sized” is meant a particle size in the nanogram range. A nanogram is 10 to the minus 9 grams (0.000000001 g). The low glycemic nano-sized glycoside used in the present invention is manufactured under a proprietary process by Nutrilab, Inc., St. Petersburg, Fla. and commercially made available by that company under the trademark TRUTINA DULCEM™. The designation BLIND AMINO ACID™ also is a trademark of the aforementioned Nutrilab, Inc.

The term “low glycemic” means a food sugar that measures less than about 50 on the Glucose Scale. According to the manufacturer, the TRUTINA DULCEM glucoside component used in the present invention is a very low glycemic and carries the seal of approval from the Glycemic Research Institute, Washington, D.C. All sugars, carbohydrates and foods have a glycemic response in the human body. Glucose has a glycemic index of 100 which creates a significant increase in blood sugar and insulin. In contrast, the glycemic index of 40 grams of TRUTINA DULCEM glycoside measures 19, that is one serving (1 gram) will not elicit any significant glycemic response in individuals taking the additive orally. Accordingly, the TRUTINA DULCEM glycoside employed in the present invention is biodegradable and not harmful to any living plant or carbon-based form.

It will be appreciated that other ingredients may be added to the active ingredient to increase efficacy such as, for example, Vitamins selected from the group consisting of Vitamins A, B, C, D, and E. Vitamin C is a particularly preferred additive, but any of the foregoing Vitamin additive or mixtures thereof may be mixed with the L-arginine and the low glycemic glycoside compound before or during the process of mixing with a suitable cosmetic base.

In accordance with the present invention, the active ingredient comprising an amino acid (L-arginine), a low glycemic glycoside, and the Vitamin component is contained in an effective amount in an acceptable cosmetic base or carrier for topical application on the surface of the skin. More particularly, the preferred cosmetic base of the present invention comprises a lip color or lip stain product comprising a water-gel base, a coloring agent, humectant, preservatives, thickeners, oils and the like. The techniques of forming such compositions are well known in the art of cosmetology. Illustrative lipcare cosmetic compositions are disclosed for example in U.S. Pat. Nos. 4,935,228; 5,288,482; and 5,879,684; the contents of which are hereby incorporated herein by this reference.

To even further illustrate the present invention without limitation, a lip stain cosmetic product was prepared containing an active ingredient comprising a mixture of L-arginine, TRUTINA DULCEM™ low glycemic glycoside, and a Vitamin, which active ingredient then was dispersed in an acceptable cosmetic base having the following formula as set forth in TABLE I below. The amount of the active ingredient was about 1.5% by weight of the total composition. The Vitamin component was present in the active ingredient mixture in an amount by weight of about 15% whereas the L-arginine and TRUTINA DULCEM™ low glycemic glycoside components made up the remainder of the active ingredient mixture by weight in a ratio of about 1:1.

TABLE 1

| INCI NAME | CLASS | % RANGE | FUNCTION |
|---|---|---|---|
| Propylene Glycol | A | 10-50% | Humectant |
| Water (Aqua) | A | 10-50% | Carrier |
| Silica | A | 5-25% | Thickener |
| PPG-26-Buteth-26 | A | 1-10% | Emulsifier |
| Flavor | B | <1% | Masking Agent |
| Polyacrylamide | B | <1% | Gelifying Agent |
| Ethylhexyl Palmitate | B | <1% | Active (Maxi-Lip) |
| PEG-40 Hydrogenated | B | <1% | Emulsifier |
| Castor Oil | | | |
| Cocos Nucifera (Coconut) | B | <1% | Active (Monoi Oil) |
| Oil | | | |
| C13-14 Isoparaffin | B | <1% | Gelifying Agent |
| Phenoxyethanol | B | <1% | Preservative |
| Tribehenin | B | <1% | Active (Maxi-Lip) |
| Red 33 (Cl 17200 | C | <1% | Pigment |
| Laureth-7 | B | <1% | Gelifying Agent |
| Yellow 5 (Cl 19140) | C | <1% | Pigment |
| Methylparaben | B | <1% | Preservative |
| Gardenia Tahitensis (Tiare) | B | <1% | Active (Monoi Oil) |
| Flower | | | |
| Sorbitan Isostearate | B | <1% | Emulsifier |
| Red 40 (Cl 16035) | C | <1% | Pigment |
| Blue 1 (Cl 42090) | C | <1% | Pigment |
| Butylparaben | B | <1% | Preservative |
| Ethylparaben | B | <1% | Preservative |
| Propylparaben | B | <1% | Preservative |
| Isobutylparaben | B | <1% | Preservative |
| Palmitoyl Oligopeptide | B | <1% | Active (Maxi-Lip) |
| Tocopherol | B | <1% | Active (Vitamin E) |
| Glycine Soja (Soybean Oil) | B | <1% | Active (Monoi Oil) |

The active ingredient comprising a mixture of amino acid (L-arginine), low glycemic glycoside and a Vitamin and the ingredients set forth in the formula of Table 1 were mixed together to form the preferred lip stain product of the invention as set forth in the following illustrative EXAMPLE 1.

### EXAMPLE 1

The L-arginine in powder form was added to the TRUTINA DULCEM™ nano-sized powder and mixed with water to produce a homogenous aqueous dispersion. The Vitamin component was then added. The colorants then were added with continued mixing followed by the humectant. The gellifier components were then added to the mixture to produce a gel phase. In a separate vessel, the emulsifier ingredients and other active ingredients were mixed together and heated to a range of between 70°Cent. and 75°Cent. In still another separate vessel, the preservatives and flavor ingredients were mixed together with a humectant and then this mixture added to the heated emulsifier mixture and stirred until homogeneous. The combined heated homogeneous mixture then was added to the gel phase and mixed until homogeneous. Finally, the thickener was added to the mixture with continued stirring until homogeneous and allowed to cool to room temperature. The resulting lip stain gel then was filled into suitable applicators.

Many modifications of the present invention will be apparent to those skilled in the art. Accordingly, the present invention should be limited only by the spirit and scope of the present invention as defined in the claims hereof.

What is claimed as being new and desired to be protected by Letters Patent of the United States is as follows:

1. A cosmetic composition suitable for topical application to human or animal skin comprising an effective amount of active ingredient dispersed in an acceptable cosmetic base or carrier, said active ingredient comprising a mixture of an amino acid, a low glycemic glycoside, and a Vitamin selected from the group consisting of Vitamin A, B, C, D, or E.

2. The composition of claim 1 wherein said amino acid is L-arginine.

3. The composition of claim 1 wherein said low glycemic glycoside is derived from kiwi fruit.

4. The composition of claim 1 wherein said amino acid is L-arginine and said low glycemic glycoside is derived from kiwi fruit.

5. The composition of claim 1 wherein said active ingredient is dispersed in said acceptable cosmetic base or carrier in an amount ranging form about 0.5% to about 3.0% by weight of the total composition.

6. The composition of claim 5 wherein said active ingredient is dispersed in said acceptable cosmetic base or carrier in am amount by weight of about 1.5% of the weight of the total composition.

7. The composition of claim 6 wherein said amino acid and said low glycemic glycoside are contained in said composition in a weight ratio of about 1:1.

8. The composition of claim 1 wherein said amino acid and said low glycemic glycoside are contained in said composition in a weight ratio of about 1:1.

9. The composition of claim 2 wherein said L-arginine and said low glycemic glycoside are contained in said composition in a weight ratio of about 1:1.

10. The composition of claim 3 wherein said amino acid and said low glycemic glycoside derived from kiwi fruit are contained in said composition in a weight ratio of about 1:1.

11. The composition of claim 4 wherein said L-arginine and said low glycemic glycoside derived from kiwi fruit are contained in said composition in a weight ratio of about 1:1.

12. The composition of claim 1 wherein said low glycemic glycoside is in the form of nano-sized particles.

13. The composition of claim 2 wherein said low glycemic glycoside is in the form of nano-sized particles.

14. The composition of claim 3 wherein said low glycemic glycoside derived from kiwi fruit is in the form of nano-sized particles.

15. The composition of claim 4 wherein said low glycemic glycoside derived from kiwi fruit is in the form of nano-sized particles.

16. The composition of claim 5 wherein said low glycemic glycoside is in the form of nano-sized particles.

17. The composition of claim 6 wherein said low glycemic glycoside is in the form of nano-sized particles.

18. The composition of claim 7 wherein said low glycemic glycoside is in the form of nano-sized particles.

19. The composition of claim 8 wherein said low glycemic glycoside is in the form of nano-sized particles.

20. The composition of claim 1 wherein said acceptable cosmetic base or carrier is a lip color composition comprising an aqueous gel phase and said active ingredient is dispersed in said gel phase.
